(19) United States
(12) Patent Application Publication
(10) Pub. No.: US 2026/0240668 A1
(43) Pub. Date: Aug. 20, 2026

(54) ADJUSTABLE SOCKET HAVING A FABRIC-BASED PANEL ASSEMBLY FOR ENCLOSING A RESIDUAL LIMB

(71) Applicant: OSSUR ICELAND EHF, Reykjavik (IS)

(72) Inventors: Andrew BACHE, Reykjavik (IS); Marco STEINBERG, Reykjavik (IS); Lukas KALEMBA, Reykjavik (IS)

(21) Appl. No.: 19/164,794

(22) PCT Filed: Mar. 14, 2024
(86) PCT No.: PCT/US2024/019855

§ 371 Date: Sep. 12, 2025

Related U.S. Application Data
(60) Provisional application No. 63/490,075, filed on Mar. 14, 2023.

(51) Int. Cl. A61F 2/80; A61F 2/78

(52) U.S. Cl. CPC A61F 2/80; A61F 2002/7837

(57) ABSTRACT

A prosthetic socket is adapted to adjustably fit to a residual limb, and has a socket frame including at least one strut defining an elongate configuration and a distal end connected to a distal base; and a fabric sleeve including at least one fabric panel assembly configured to form a circumferential and a generally conical enclosure for receiving a residual limb, the at least one fabric panel assembly securing to the socket frame.

### INCORPORATION BY REFERENCE TO RELATED APPLICATIONS

The pending application incorporates herein by reference the following patents: U.S. Pat. No. 8,795,385, granted on Aug. 5, 2014; U.S. Pat. No. 9,050,202, granted on Jun. 9, 2015; U.S. Pat. No. 9,962,273, granted on May 8, 2018; U.S. Pat. No. 10,806,607, granted on Oct. 20, 2020; U.S. Pat. No. 10,828,180, granted on Nov. 10, 2020; U.S. Pat. No. 10,940,028, granted on Mar. 9, 2021; U.S. Pat. No. 10,966,851, granted on Apr. 6, 2021; U.S. Pat. No. 11,185,430, granted on Nov. 30, 2021; U.S. Pat. No. 11,547,589, granted on Jan. 10, 2023.

### TECHNICAL FIELD

The disclosure relates to an adjustable socket system for a residual limb, specifically an adjustable socket including a fabric sleeve arranged to intimately fit a residual limb within a substantially rigid or rigid and adjustable socket frame.

### BACKGROUND

A typical prosthetic leg and foot include a socket, pylon, and foot. A socket is commonly referred to as the portion of a prosthesis that fits around and envelops a residual limb or stump and to which prosthetic components, such as a foot, are attached. The fitting and alignment of the socket are difficult tasks and require extensive knowledge, training, and skill from the prosthetist.

The socket should fit closely to the stump to provide a firm connection and support but must also be sufficiently loose to allow for circulation. In combination with proper fitting, the socket must transfer loads from the residual limb to the ground comfortably.

Conventional sockets are typically rigid and may have a uniform shape that receives a substantial portion of the residual limb. These sockets are permanently formed to a customized, static shape, meaning the socket does not account for shape and volume fluctuations of the residual limb. When there are shape and volume fluctuations, the socket fitting is impeded, with these sockets causing discomfort, pain, and soft tissue breakdown of the stump. Conventional sockets also tend to be bulky and cumbersome to wear and may be difficult to don, making the residual limb uncomfortable when worn.

Attempts have been made to develop adjustable sockets with individual components that can be varied in size and shape to account for volume and shape fluctuations of the residual limb. These adjustable sockets, however, tend to have labor-intensive and complicated tightening systems for donning and doffing the socket, making their use difficult for patients with limited dexterity, cognition, and strength. These drawbacks can result in unsafe and improper socket use, causing discomfort and injury.

Conventional sockets are known to inadequately account for shape and volume fluctuations of a residual limb. They also tend to be bulky and cumbersome to wear. Known adjustable socket systems may better accommodate shape and volume fluctuations than conventional sockets but tend to be uncomfortable and provide inadequate support and suspension. Thus, an adjustable socket is needed to comfortably accommodate shape and volume fluctuations of the residual limb and provide improved support and stability.

Because of the foregoing discussion, there is a need for an improved adjustable socket system that overcomes the problems of existing sockets.

### SUMMARY

Embodiments of the present disclosure comprise an adjustable socket system that provides an intuitive and simple manner for users with limited dexterity or cognition to don and doff the system. From its straightforward and versatile design, the adjustable socket system can improve ease of use, and decrease the likelihood of over-tightening and under-tightening of the system over known adjustable socket systems.

Embodiments of the present disclosure comprise an adjustable socket system that provides superior structure adjustment features over prior art adjustable socket systems. The adjustable socket system can decrease discomfort and improve support and stability over known adjustable sockets due to its versatility in fitting and force distribution. Moreover, it will be appreciated that the adjustable socket systems of the present disclosure may be adapted to several types of amputations, whether configured for the leg or arm.

A general embodiment of the disclosure comprises a prosthetic socket adapted to adjustably fit to a residual limb. The prosthetic socket defines an axis extending centrally between proximal and distal ends of the prosthetic socket. A socket frame includes at least one strut having an elongate configuration and having a distal end connected to a distal base. A fabric sleeve includes at least one fabric panel assembly configured to form a circumferential and a generally conical enclosure for receiving a residual limb. The at least one fabric panel assembly secures to the socket frame.

In another embodiment or variation of the general embodiment, the prosthetic socket is adapted to fit to a residual limb adjustably and includes at least one fabric panel assembly configured to form a circumferential and generally conical enclosure for receiving a residual limb. The at least one fabric panel assembly secures to a socket frame, including at least one strut with an elongate configuration and a distal end connected to a distal base. At least one cable may extend through portions of the at least one fabric panel assembly and associate with the frame assembly to adjust the enclosure's fit and size by regulating a cable tensioner. Suitable cable guides and other frame elements may be incorporated with the socket frame or the at least one fabric panel assembly to guide the at least one cable and provide structure to the at least one fabric panel assembly.

### BRIEF DESCRIPTION OF THE DRAWINGS

These and other features, aspects, and advantages of the present disclosure will be better understood in the following description, appended claims, and accompanying drawings.

FIG. 1A is a frontal elevational view of an anterior side of the adjustable prosthetic socket.

FIG. 1B is a frontal elevational view of a posterior side of the adjustable prosthetic socket.

FIG. 2 is an elevational view of a socket frame in the adjustable prosthetic socket of FIG. 1.

FIG. 3 is an elevational view showing a medial side of the adjustable prosthetic socket of FIG. 1.

FIG. 4 is an elevational view showing the socket from the medial side of the frame assembly of FIG. 2 without the fabric sleeve.

FIG. 5 is an elevational view showing a lateral side of the adjustable prosthetic socket of FIG. 1.

FIG. 6 is an elevational view showing the socket from the lateral side of the frame assembly of FIG. 2 without the fabric sleeve.

FIG. 7 is a perspective view of a first fabric panel in the prosthetic socket of FIG. 1.

FIG. 8 is a perspective view taken from a distal end exemplifying the fabric panel assembly extending about the distal base in the prosthetic socket of FIG. 1.

FIG. 9 is an elevational view of a cable guide.

FIG. 10 is an exemplary view showing radial expansion of the fabric panel assembly of the prosthetic socket of FIG. 1.

FIG. 11 is an exemplary view of a stiffening rib disposed in or about the fabric panel assembly of the prosthetic socket of FIG. 1.

FIG. 12A is a front perspective view of a distal junction part in the prosthetic socket of FIG. 1.

FIG. 12B is a rear elevational view of the distal junction part of FIG. 12A.

FIG. 12C is a side elevational view of the distal junction part of FIG. 12A.

FIG. 12D is an exemplary view illustrating the cable routing on the side of the prosthetic socket.

FIG. 12E is an exemplary view of detail 12E in FIG. 5.

FIG. 13 is a schematic view showing an amputated limb versus an intact limb.

FIG. 14 is a perspective view showing the articulation of the socket frame of the prosthetic socket of FIG. 1.

FIG. 15 is a perspective view of a posterior side of the distal base in the prosthetic socket of FIG. 1.

### DEFINITIONS

Descriptions of the following terms are provided for further ease of understanding the embodiments of an adjustable socket system as disclosed herein. For explanatory purposes, each embodiment or component described herein may be divided into sections denoted by general anatomical terms for the human body. Such anatomical terms are provided to distinguish various elements of the device embodiments from one another, but which are not to be considered to limit the scope of the disclosure.

As used herein, the term “proximal” has its ordinary meaning and refers to a location closer to the heart than another. Likewise, the term “distal” has its ordinary meaning and refers to a location that is further from the heart than another location. The term “posterior” also has its ordinary meaning and refers to a location that is behind or to the rear of another location. The term “anterior” has its ordinary meaning and refers to a location that is ahead of or to the front of another location.

The terms “inwardly” or “inner” commonly used herein to distinguish the side of the device that may be directed to the posterior side of the device and specifically adjacent to the residual limb of the wearer of the device or directed to the central axis of the residual limb. Contrariwise, the term “outwardly” or “outer” are used to denote the side of the device that is opposite to the inwardly side.

The terms “medial” and “lateral” are relative terms that are understood as indicating location with respect to the midsagittal plane or midline. Therefore, elements that are located near the midline are referred to as “medial” and those elements that are further from the midline are “lateral.” The term “central” is used to denote the area along the midline of a joint thereby dividing and sharing regions of the medial and lateral regions.

The terms “substantial” or “substantially” means at minimum a figure or degree greater than 50 percent up to and including 99.9 percent. The term “general” means broad or broadly based, whereas “generally” means mostly or closely related.

The terms “rigid,” “flexible,” and “resilient” may be used herein to distinguish characteristics of portions of certain features of the prosthetic system. The term “rigid” is intended to denote that an element of the device is devoid of flexibility. On the other hand, the term “flexible” is intended to denote that features are capable of repeated bending such that the features may be bent into retained shapes or the features do not retain a general shape, but continuously deform when force is applied. The term “resilient” is used to qualify such flexible features as generally returning to an initial general shape without permanent deformation. As for the term “semi-rigid,” this term is used to connote properties of members that provide support and are free-standing; however, such members may have flexibility or resiliency. The term “stretch” means being made or capable of being made longer or wider without tearing.

The term “cable” as used herein means cable, lace, or wire. The cable may be an elongated wire-like element made of uniform material or of material arranged as a strand.

The term “fastener” refers to any suitable connecting or tightening mechanism or structure expressly including, but not limited to, rivets, screws, bolts and the combinations of bolts and nuts (e.g., without limitation, lock nuts) and bolts, washers, and nuts.

The term “strut” means a rigid, semi-rigid, elongated structural element forming part of a framework and designed to resist compression.

### DETAILED DESCRIPTION OF VARIOUS EMBODIMENTS

A better understanding of different embodiments of the disclosure may be had from the following description read with the accompanying drawings in which reference characters refer to like elements.

While the disclosure is susceptible to various modifications and alternative constructions, certain illustrative embodiments are in the drawings and are described below. It should be understood, however, that there is no intention to limit the disclosure to the specific embodiments disclosed, but on the contrary, the intention covers all modifications, alternative constructions, combinations, and equivalents falling within the spirit and scope of the disclosure.

It will be understood that unless a term is expressly defined in this application to possess a described meaning, there is no intent to limit the meaning of such term, either expressly or indirectly, beyond its plain or ordinary meaning.

Any element in a claim that does not explicitly state “means for” performing a specified function, or “step for” performing a specific function is not to be interpreted as a “means” or “step” clause as specified in 35 U.S.C. § 112(f).

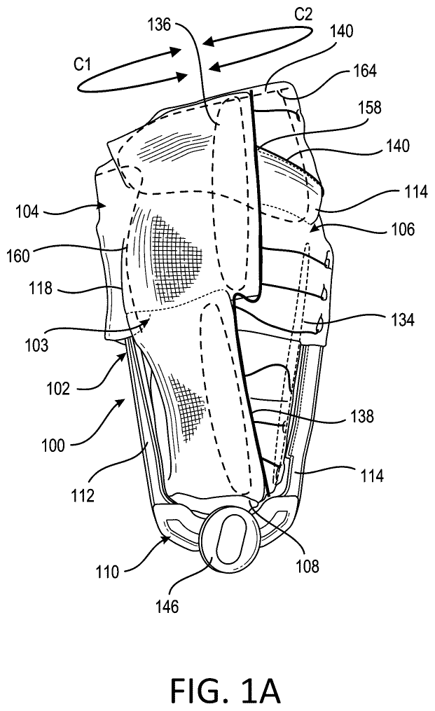

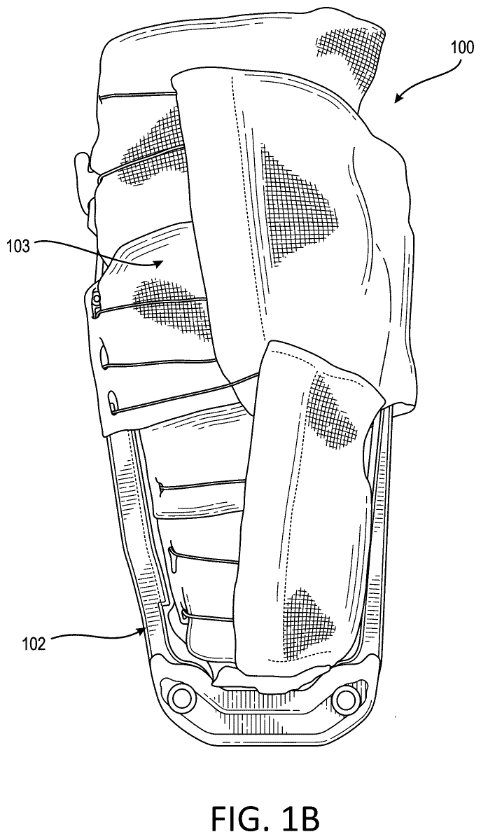

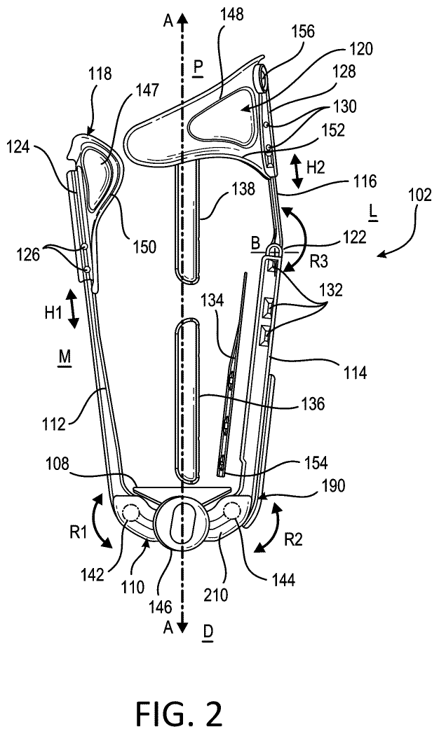

In observing FIGS. 1A-2, a prosthetic socket 100 is adapted to fit to a residual limb adjustably. It is therefore considered an “adjustable socket” as compared to a rigid socket having a predetermined size and configuration, conventionally known in the art of prosthetic devices.

The prosthetic socket 100 includes a socket frame 102 including at least one strut 112, 114 having an elongate configuration and having a distal end connected a distal base 110. A sleeve 103 formed at least one fabric panel assembly of fabric panels 104, 106 is configured to form a circumferential and generally conical enclosure for receiving a residual limb. The sleeve 103 is secured to the socket frame 102 yet can be individually sized relative to the socket frame 102 and is arranged to secure the limb about a central axis A-A of the prosthetic and within the socket frame 102.

The sleeve 103 is arranged to provide an intimate fit to the residual limb and can accommodate a variety of individually sized and configured residual limbs. The sleeve 103 defines a proximal circumference or circumferential segment C1, C2 adapted to adjust in size upon donning and resilient return to be urged to return to a predetermined configuration. The first proximal circumference segment C1 is defined about the medial side of the sleeve 103 and the second proximal circumference segment C2 is defined about the lateral side of the sleeve 103. While the at least one strut or first and second struts 112, 114 are substantially rigid, the at least one fabric panel assembly of fabric panels 104, 106 are flexible.

According to the embodiment of FIGS. 1A-2, the sleeve 103 includes a first fabric panel 104 and a second fabric panel 106. Portions of the first and second fabric panels 104, 106 may be inelastic and elastic, depending on the location relative to the socket frame 102 or a tightening mechanism to be discussed below. Localized regions of the first and second fabric panels 104, 106 can be knitted such that the material of the first and second fabric panels 104, 106 adapts to regions of high and low pressure. The first and second fabric panels 104, 106 may be movable relative to one another and connected to one another by at least one cable 140 permitting tensioning to secure and support a residual limb firmly. The first and second fabric panels 104, 106 may overlap one another to continuously encircle the residual limb, thereby forming the fabric sleeve, such that a peripheral edge 158 of one of the first and second panels 104, 106 extends from the distal end to a proximal end of the prosthetic socket 100, and over the other fabric panel.

Preferably, the fabric is formed by at least one textile or textile stitch, knit or pattern. The fabric should conform to a residual limb but may not stretch, at least in the sleeve, as it would increase socket volume and affect socket fit and comfort. The textile can be created using a knit or a weave. Alternatively, however, the fabric could be arranged to stretch in varying degrees, as the disclosure and the embodiments herein are not limited to a stretch less textile.

By providing a sleeve formed from a fabric comprising a textile or plurality of textiles, the prosthetic socket can provide the advantages of better comfort over conventional adjustable prosthetic sockets, especially seated comfort. Because the fabric-based sleeve is formed of textiles, there is a significant cost reduction of materials, such as eliminating a need for many injection molded plastic components, and therefore makes it easier to scale commercial products and enable quicker iterations of sleeve configurations by knitting or stitching, as opposed to making plastic molds. The fabric sleeve offers a more lightweight configuration over plastic components and enables a more natural interface as the human body is more accustomed to wearing textile-based garments than plastic structures. The fabric sleeve, therefore, offers an improved and more natural appearance that is less bulky and provides better clothing compatibility.

The fabric sleeve may be configured and formed according to any of the principles according to U.S. Pat. No. 10,966,851, incorporated by reference. Low-stretch and high-durability yarns may be used to form the fabric sleeve. These yarns may be composed of substances such as Kelvar or Dyneema/Spectra and can be selected for prosthetic applications as they conform but do not stretch.

Low-melt yarn combined with yarn may likewise be used. “Grilon Thermoformable Yarn” is an example of a low melt plastic it is knitted in conjunction with another yarn such as polyester. The sample is then placed on a steam bed to melt the Grilon. This allows the creation of a piece of textile that does not stretch and is robust. If one were to try to knit a textile piece with the required level of stretch and robustness from pure plastic and textile the knitting machine would unlikely be able to knit it.

Another method of creating a non-stretch textile is to knit from a hot-melt yarn (such as TPE or T.P.U.). This knitted plastic piece is then melted (in a hot press for example) during the thermal textile finishing process and stiffens or stabilizes the surface. It is also possible that the knitted plastic piece is placed into a shaped tool such that when heated in the shaped tool, the knitted part melts into a desired shape. It is possible to have combinations of melted yarn and other yarn. This arrangement permits the optimal combination of stiffer and structural elements, and softer and comfort elements.

Yarns may be coated for hydrophobicity to prevent sweat from penetrating into the socket. This improves hygiene. It is possible to also coat the complete part after knitting. Antibacterial yarns can also be used, for example, silver. Moisture sensitive yarn can be used as absorption of moisture causes yarn to shorten or lengthen. This can be used to increase size of openings in the knit, thus controlling the flow of air and moisture through the fabric. Conductive yarns can create electrical pathways, switches, and E.M.G. sensing.

Amputees wish to have a black or dark-colored socket or other prosthetic device, which draws less attention. The disadvantage of dark colors is that they heat up easily. This is why it is common, for example, for an amputee to cover their leg up with a towel when on the beach. It is possible to use yarn, which even though black and dark in color, absorbs less and reflects infrared thermal radiation. Luminescent yarn is charged when exposed to light and can have a significant afterglow. Glowing or light-reflective yarn can be used to aid an amputee in going to the toilet in the middle of the night. Heating elements such as electrically conductive wires can be used to heat the socket, as it is desirable to improve comfort. For example, it can be unpleasant to put on a cold prosthesis. Conversely, cooling elements can also be knitted into the fabric structure to improve comfort in response to warmer environments and settings.

Knitting or weaving may be selected to provide an advantageously improved fabric sleeve. While the fabric sleeve may be arranged to resist stretching or stretch only in a predetermined location, localized regions may be knitted so that the textile adapts to high-and low-pressure regions. A plush or pile weave (i.e., a compactly woven structure with a large pile) permits a range of useful features, such as effecting rotational control, axial movement or pistoning, and creating or enhancing suspension.

Additional features, like a color change kit, may be associated with the knit or weave. For example, a first knit with a first color may be underneath a second knit with a second color. When force is applied to the second knit, the first knit may be exposed and thus its color. Multiple layers of different colored fabrics may be used and exposed at different pressure levels.

The fabric sleeve may be provided with an inner liner or layer formed from an elastomeric polymer, or regions may be provided with elastomeric polymer to resist sliding and provide a more rigid or anti-slipping interface with the skin of the residual limb. Such areas of an elastomeric polymer may be selectively formed to resist pistoning, improve durability or other desirable properties.

Electrodes can move or be positioned by moving them relative to the stump by to attain an optimal signal. This could be achieved using a pocket or push fit for example. Electrodes may be spring-loaded, such as by holding them in position against the skin. The spring may be a metal compression spring or other type of material spring.

Concerning the socket frame 102, the prosthetic socket 100 defines the central axis A-A extending between proximal and distal ends P, D of the prosthetic socket 100, and about which the at least one strut 112, 114 is arranged to pivot relative thereto. The at least one strut 112, 114 includes a first strut 112 generally extending on a lateral side L of the axis A-A and a second strut 114 generally extending on a medial side M of the axis A-A.

The distal end of the first and second struts 112, 114 may be pivotally connected to a distal base 110 and adapted to rotate R1, R2 relative thereto at at least one pivot point 142, 144 corresponding to each of the first and second struts 112, 114. The first and second struts 112, 114 are secured once adjusted to fit a residual limb. For example, the at least one pivot point 142, 144 is lockable by a fastener extending through the distal base 110 and engaging at least one of the first and second struts 112, 114.

A funnel 108 is secured to the distal base 110. A distal lock 146 connects to the distal base 110 and is adapted to fit to a prosthetic pin and couple to a prosthetic system. An example of a distal base 110 is provided in U.S. Pat. No. 11,185,430 incorporated herein by reference.

As shown in FIGS. 2 and 4, a medial piece or shell 118 connects to a proximal end of the first strut 112. The medial piece 118 defines at least one wing, and more specifically, opposing wings extending laterally relative to the first strut 112. The medial piece 118 is flexible or semi-flexible relative to the first strut 112. The first strut 112 is substantially or entirely rigid. The at least one wing 147 has a peripheral shape 150 that tapers away or reduces in height relative to the first strut 112.

The medial piece 118 defines a mounting portion 124 slidably attached to the proximal end of the first strut 112 by a channel (not shown) defined by the mounting portion 124 and is configured and dimensioned to a cross-section of the first strut 112. The medial piece 118 is adapted by the mounting portion 124 to adjust in height H1 relative to the first strut 112. The medial piece 118 is secured along a plurality of locations relative to the proximal end of the first strut 112 by at least one fastener 126 arranged to lock the medial piece 118 and the first strut 112.

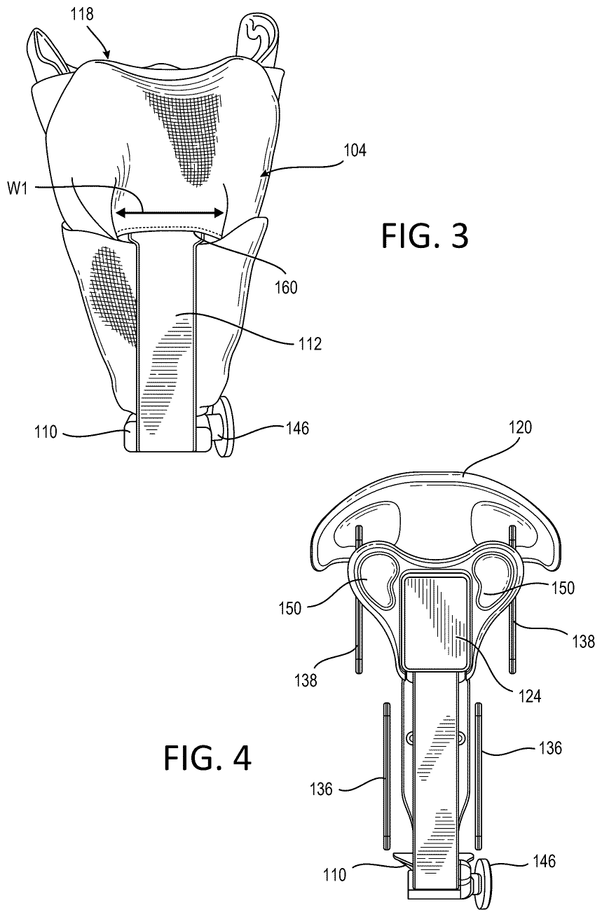

FIG. 3 illustrates that the first fabric panel 104 is adapted to fit to or about the medial piece 118. The first fabric panel 104 defines a first pocket 160 configured and dimensioned to receive the medial piece 118 and elastically expand a width W1 to receive the medial piece 118 and secure the first fabric panel 104 at least about the medial piece 118. In an alternative or in supplement to the pockets, adhesives or fasteners may be used to secure the medial piece to the first fabric panel 104, or the fabric sleeve in general. In yet another alternative, the fabric sleeve may be stitched, riveted, or glued about the medial piece such that it is embedded within the sleeve or at least partially embedded therein.

FIGS. 2, 6 and 14 illustrate at least one strut 112, 114, including a third strut 116 hingedly secured about an axis B-B by a hinge 122 extending perpendicularly relative to the central axis A-A.

A lateral piece or shell 120 is connected to a proximal end of the third strut 116. The lateral piece 120 defines at least one wing 148, preferably a pair of wings, extending laterally relative to the third strut 116. The lateral piece 120 is flexible or semi-flexible relative to the second and third struts 114, 116. The second and third struts 114, 116 are substantially or entirely rigid. As in the medial piece 118, at least one wing 148 of the lateral piece 120 has a peripheral shape 152, tapering away or reducing in height relative to the third strut 116.

The lateral piece 120 defines a mounting portion 128 slidably attached to the proximal end of the second strut 114 by a channel (not shown) defined by the mounting portion 128 and configured and dimensioned to a cross-section of the second strut 114. The lateral piece 120 is adapted by the mounting portion 124 to adjust in height H2 relative to the third strut 116. The lateral piece 120 is secured along a plurality of locations relative to the proximal end of the third strut 116 by at least one fastener 130, locking the lateral piece 120 and the third strut 116.

The fasteners 126 or set screws in the socket frame 102 permit a low-profile solution important for the medial aspects of the socket, which can rub onto the contralateral leg creating discomfort. It is also generally important to the overall appearance, which should be as low profile to better fit into clothing or be less noticeable.

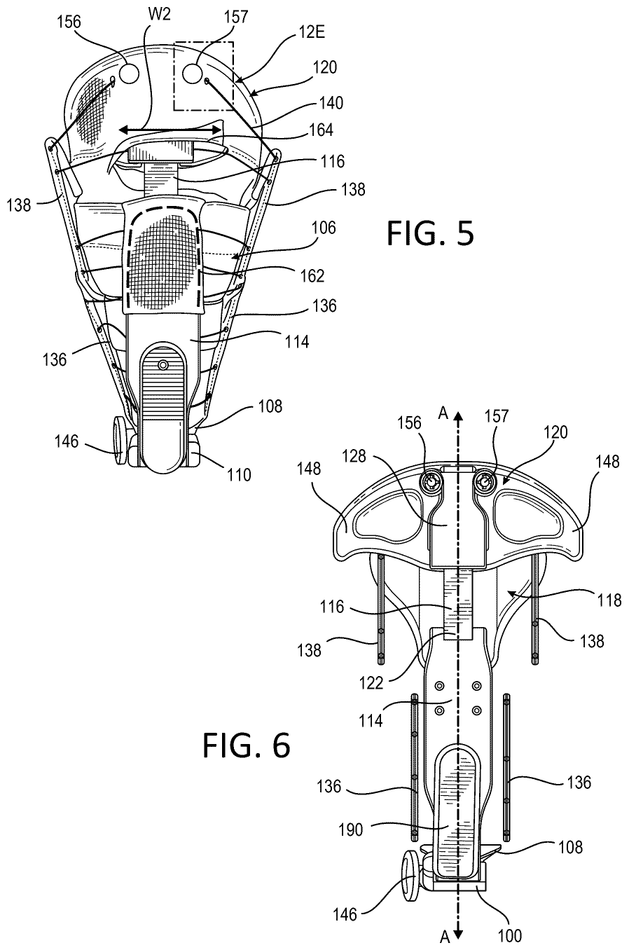

FIG. 5 shows that the second panel 106 is adapted to fit about a portion of the second strut 114. The second panel 106 defines a second pocket 162 configured and dimensioned to receive a portion of the second strut 114. Further, the second panel 106 is adapted to fit about the lateral piece 120. The second panel 106 defines a third pocket 164 configured and dimensioned to receive the lateral piece 120 and elastically expands a width W2 to receive the lateral piece 120 and secure the second panel 106 at least about the lateral piece 120. As an alternative or in supplement to the pockets, adhesives or fasteners may be used to secure the lateral piece to the second fabric panel 106, or the fabric sleeve in general. In yet another alternative, the fabric sleeve may be stitched, riveted, or glued about the lateral piece such that it is embedded within the sleeve or at least partially embedded therein.

The medial and lateral pieces or shells 118, 120 may be formed by a polymeric material to offer a more compatible interface than the struts 112, 114, 116, which are formed from a rigid structural material, to connect to weight-bearing structures and allowing tensioning. Both the medial and lateral pieces 118. 120 are shaped to provide both comfort and support by the extension of the lateral wings 147, 148, and their corresponding peripheral shape 150, 152 tapering laterally. Accordingly, by adjusting their heights relative to the struts, the medial and lateral pieces can be telescoped up and down in height relative to the distal end of the first and third struts 112, 116 to accommodate different limb lengths.

The medial and lateral pieces may be of a polymeric material such as resilient thermoplastic elastomer (TPE) to provide a soft transition to the residual limb in combination with their structural features. Likewise, the medial and lateral pieces may be composed of a rigid plastic material, such as polypropylene (PP) or Acrylonitrile Butadiene Styrene (ABS) over-molded with a polymeric material such as TPE, as taught in U.S. Pat. No. 11,547,589, and incorporated herein by reference. Indeed, the medial and pieces may be provided similarly to any of the shell embodiments of U.S. Pat. No. 11,547,589. The medial and lateral pieces may contain metal inserts to allow set screws to lock them in place relative to the medial and lateral struts.

As noted above, the prosthetic socket 100 may further comprise a cable tensioner or tensioning unit 190 coupled to at least one cable 140 extending between the first and second struts 112, 114 and about the sleeve 103. The cable tensioner 190 is adapted to control the tension of at least one cable 140 about at least a segment or entirely of a circumference of the sleeve 103.

The cable tensioner 190 and the at least one cable 140 may be arranged similarly as in any of the embodiments of U.S. Pat. No. 10,940,028. Alternative examples of the cable tensioner 190 are provided in U.S. Pat. Nos. 8,795,385 and 9,050,202.

To better route the at least one cable 140 about the socket frame 102 and the fabric sleeve, the second strut 114 may define at least one cable passageway 132 extending through a width of the second strut 114. Further, the sleeve 103, and more appropriately, either of the first and second fabric panels 104, 106 may include at least one cable guide 136, 138. The at least one cable guide 136, 138 may be formed by guides formed or stitched to the fabric panels, or a separately formed cable guide, defined as a plate, such as an elongate plate in FIGS. 2 and 9, secured to or embedded within the at least one fabric panel assembly 104, 106. The at least one cable guide 136, 138 defines at least one opening 188 to a channel 186 through which the at least one cable 140 routes or extends.

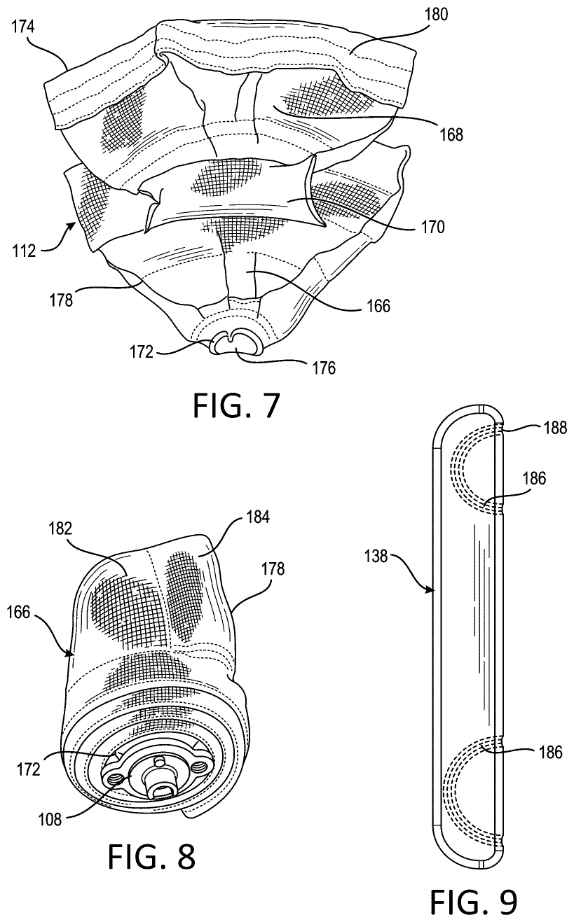

FIGS. 7 and 8 illustrate an exemplary and non-limiting configuration of the first fabric panel 104, and the second fabric panel 106 may be similarly constructed or adapted accordingly. The first fabric panel 104 comprises a distal fabric subpanel 166, a proximal fabric subpanel 168, and an elastic subpanel 170 connecting the distal and proximal fabric subpanels 166, 168. The elastic subpanel 170 has greater elasticity than the distal and proximal fabric subpanels 166, 168, so the distal and proximal fabric subpanels are preferably inelastic.

The distal fabric subpanel 166 defines a distal edge 172 bordering an enclosed opening 176 elastically or inelastically adapted to fit about the funnel 108 or distal base 110. The distal fabric subpanel 166 defines a peripheral edge 178 extending or commencing a distance spaced from the enclosed opening 176 at a distal end of the first fabric panel 104 and extending to at least the first and second sides of a distal end of the elastic subpanel 170.

FIG. 8 shows how the at least one fabric panel assembly 104, 106, such as the first fabric panel, may have its distal subpanel encircle the funnel 108 or distal base such that the funnel 108 extends within the enclosure formed by the at least one fabric panel assembly 104, 106.

Most transfemoral amputees are sensitive to pressure in the medial proximal aspect of the socket. Additional padding can be placed there to improve comfort. The padding can be created as part of the knitted part, or a pocket can be made in the textile part and padding inserted into it. Like the medial proximal aspect, the lateral proximal aspect can be a source of discomfort. Padding placed at the lateral proximal aspect can increase comfort.

There are known regions of a prosthetic socket that can cause discomfort, such as fibula head or the distal end of bone (femur/tibia). These regions can be padded accordingly. It is possible to allow the CPO (Certified Prosthetist Orthotist) or end user to optimize the padding in an area. Distinct types or degrees or layers of padding can be applied if a pocket is used. Too much padding may be perceived as bulky, cumbersome, ugly, or unstable; too little padding may create discomfort. Padding may be non-Newtonian fluid or gel, such that viscosity changes under stress. This would benefit from being soft and pliable when sitting, relaxed and stiffer, and having more support when walking and being more active. The non-Newtonian material may be the yarn, which is knitted into the structure.

FIG. 7 exemplifies the possibility of the aforementioned padding. The proximal fabric subpanel 168 defines a proximal edge 174 and a padding band 180 at, proximate, or adjacent to the proximal edge. The padding band 180 defines a padding element (not shown) embedded or attached to an interior side of the proximal fabric subpanel 168.

FIG. 8 illustrates how the first fabric panel 104 or fabric sleeve may include at least two regions 182, 184 of first and second fabric configurations or composition, according to any of the aforementioned textile or fabric possibilities.

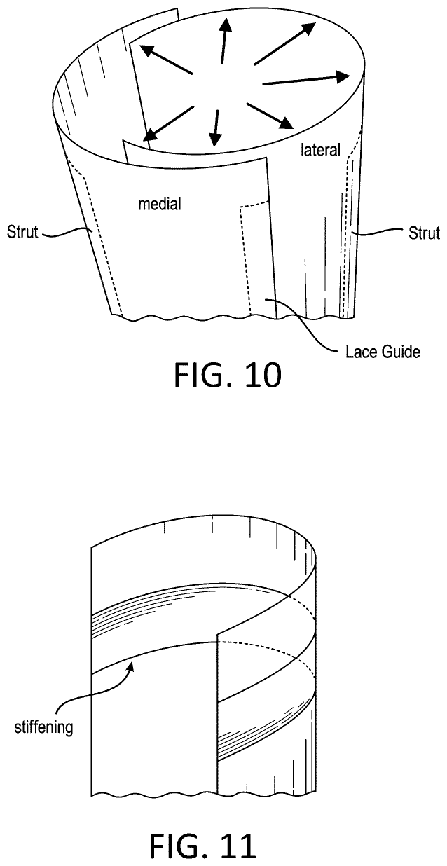

FIG. 10 exemplifies radial expansion of the fabric sleeve. It is desired that the fabric sleeve does not sag, crease, or deform internally as this will make donning difficult and compromise loading. For example, the liner locking pin should not be obstructed when moving through the socket into the distal end or lock. As the amputee pushes into the socket and applies circumferential tensioning or radial forces, the fabric shells must be prevented from creasing or deforming and creating peak pressure or suboptimal loading.

FIG. 11 illustrates the possibility of providing a stiffening pocket and stiffening plate to the fabric sleeve. It is possible to allow the CPO or end user to optimize the stiffening in an area. Diverse types or degrees of stiffening can be applied if a pocket is used. Too much stiffening may be perceived as uncomfortable. Too little stiffening could create issues with donning and load distribution. It can be a separate piece, for example, a plastic piece inserted into a textile pocket.

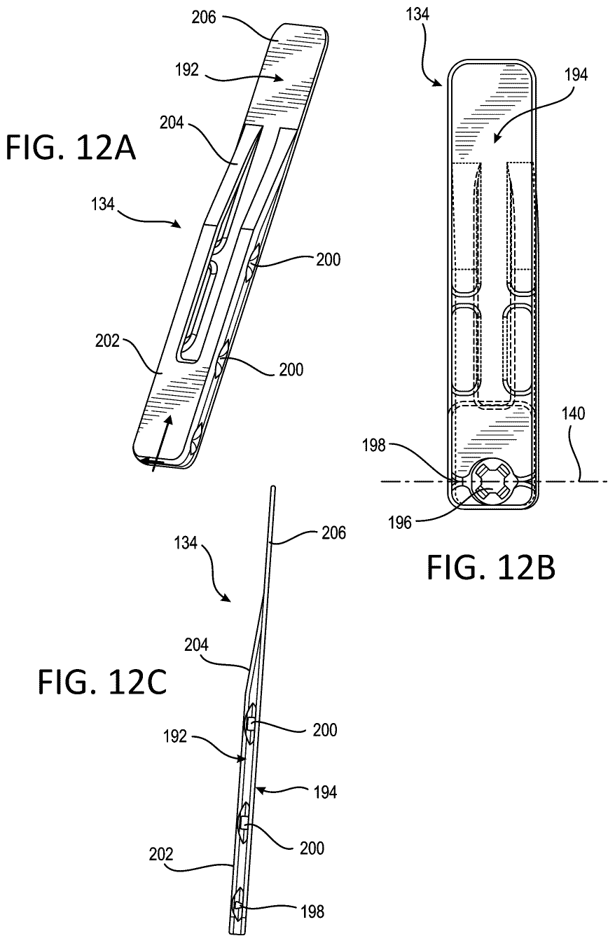

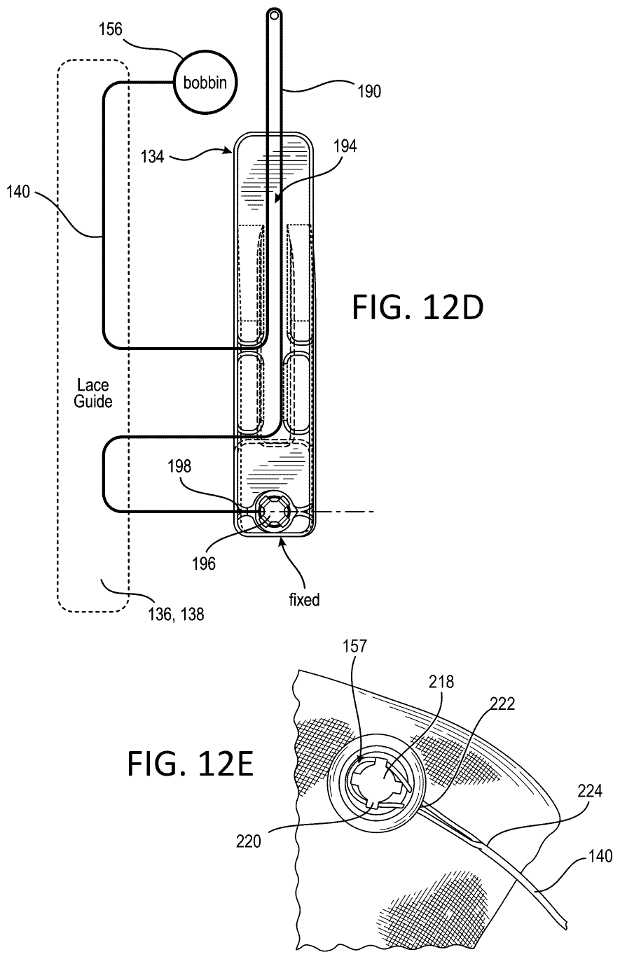

FIGS. 12A-12C show a junction part 134 secured to or embedded in the at least one fabric panel assembly 104, 106. The junction part 134 has a distal junction 154 receiving the at least one cable 140 from the cable tensioner 190. The junction part 134 has at least one passageway 198, 200 through which the at least one cable 140 extends. The junction part 134 is adapted to extend on the lateral side L of the prosthetic socket and may be embedded in the at least one fabric panel assembly 104, 106. The at least one cable 140 extends from the cable tensioner 190 and is received by a distal junction or anchor 196 formed by or associated with the junction part 134 and enables routing of and fixes the at least one cable 140 to extend laterally therefrom relative to the central axis A-A.

The junction part 134 forms a residual limb facing side 192 and an away side 194 opposite the residual limb facing side 192. The junction part 134 forms a distal section 202, a proximal section 204, and a middle section 206. The distal section 202 has a first thickness greater than a second thickness of the proximal section 206. The middle section 204 tapers from the first thickness to the second thickness along the front or limb facing side 192 to accommodate a shape of a residual limb due to the flexibility of the thinned proximal section 206 such that the rear or away side 194 is substantially flat or entirely flat continuously between the distal and proximal sections 202, 206.

The lateral part 120 may include at least one proximal anchor 156, 157 for receiving a proximal end of the at least one cable 140, as shown in FIG. 6. Referring briefly to FIG. 6, the proximal anchor 156 is located on a slightly anterior side of axis A-A, and the proximal anchor 157 is located on a slightly posterior side of axis A-A.

The junction part is provided with, in or on the fabric sleeve to allow the cable connection via the cable tensioner and socket frame, as intimate as possible rather than creating space between the socket frame and the fabric sleeve to accommodate a junction. The fabric sleeve and the socket frame should be arranged significantly close to one another with minimization of gaps and clearances. By contouring the tapered thickness of the junction part, in combination with the cable passageways, the cable is enabled to follow the shape of the residual limb closely.

FIG. 12D is an exemplary view showing how the at least one cable 140 may extend from the junction part 134, feed through the cable guides 136, 138, couple to the cable tensioner 190, and extend to the proximal anchor 156. Each side of the socket may include a cable routed in the manner shown in FIG. 12D. The junction part 134 preferably provides a distal anchor for at least two cables (i.e., arranged for first and second sides of the socket, as shown in FIG. 12D), provides channels for the cables, and may be inserted into a pocket defined by the panel assembly.

FIG. 12E details the posterior proximal anchor 157, such that the anchor 157 receives the cable 140 looped about a central post 218 and retained thereabout by retainers 220. The anchor 157 defines an aperture 222 through which the cable 140 extends. A ferrule 224 or other cable tie may secure the loop formed by the cable 140. The anterior proximal anchor 156, and the distal anchor 196 may be similarly formed, although the distal anchor may have opposed apertures adapted for two cables or cable segments to extend therefrom.

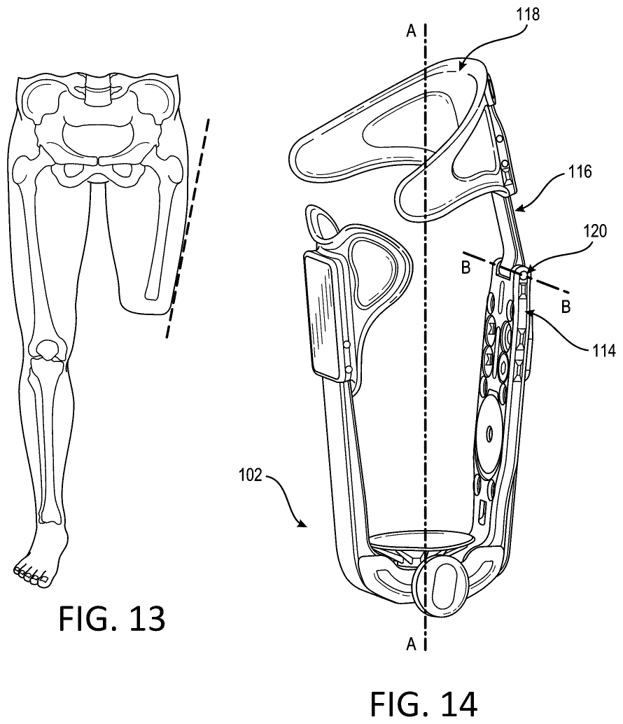

Referring to FIGS. 13 and 14, a problem with conventional adjustable sockets is that the proximal lateral region may be incongruent with the trochanter or hip of the user, which leaves an unwanted pocket or gap between the socket and the user. Being incongruent to the body in this area creates the following issues: decreased control of residual limb; decreased proprioception of socket or prosthesis; decreased comfort (a socket should have as large a surface area as possible to distribution pressure maximally); and decreased cosmesis (this pocket is readily visible even under clothing, particularly when walking). By providing the third strut 116, which is hingedly mounted to the second strut 114 at the hinge 122, and further with the lateral part 120 that includes flexible or semi-flexible wings, at least compared to the rigidity of the second strut 114, the socket frame 102 can provide better congruence to the hip or limb of the user.

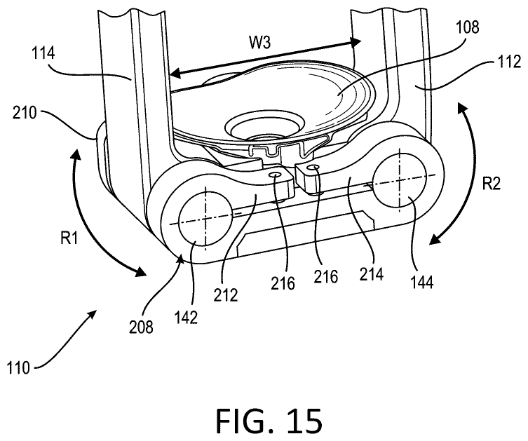

FIG. 15 illustrates an embodiment of the distal base 110. A funnel 108 enables interconnection between a liner or sleeve 103 and the prosthetic socket 100, the funnel 108 being secured to the distal base 110. The first and second struts 112, 114 are pivotally connected to the distal base 110 and adapted to rotate R1, R2 relative thereto at at least one pivot point 142, 144 corresponding to each of the first and second struts 112, 114. The pinch clamp formed by the pinch plates 212, 214, for example, positions the point of rotation much closer to the midline, allowing the width of the distal end to be narrower, which can become important when using a fabric sleeve that has a much lower profile and again better congruency to the residual limb. It is desired to reduce the incongruency and any gaps or clearances, thereby providing a close and proximate configuration of the socket frame versus the fabric sleeve.

The distal base 110 comprises pinch plates 212, 214 at an anterior part 208 of the distal base 110. Pinch plates may also be formed at a posterior part 210 of the distal base. The pinch plates 212, 214 encircle the pivot points 142, 144, corresponding to each of the first and second struts 112, 114. The pinch plates 212, 214 may restrict pivot point 142, 144 rotation and are secured to the distal base 110 by fasteners 216. In an embodiment, the distal base 110 utilizes friction discs to increase the friction between struts 112, 114 and the distal base 110 to prevent unwanted rotation when locked.

The distal base has a pinch clamp which permits the anterior, medial, and lateral aspects to be “clean” with no visible screws or protruding components. The pinch clamp is located posteriorly, with adjustment on the distal aspect of the distal end. The pinch clamp may yet be non-visible under a plastic overmold. The funnel can be centered between the pinch parts. The pinch parts allow the struts to be locked in place once the struts are in optimum position; however, it is also possible to alter the position of the distal end relative to the struts depending on if the CPO would prefer to alter the orientation of the knee or rest of the prosthesis relative to the socket or struts. The pinch clamp concept is a way to lock the strut in place with minimal torque.

It is understood that not all objects or advantages may be achieved under any embodiment of the disclosure. Those skilled in the art will recognize that the prosthetic socket may be embodied or conducted in a manner that achieves or optimizes one advantage or group of advantages as taught herein without necessarily achieving other objects or advantages as may be taught or suggested herein.

The skilled artisan will recognize the interchangeability of various disclosed features. In addition to the variations described herein, other known equivalents for each feature can be mixed and matched by one of ordinary skill in this art to construct a prosthetic socket under principles of the present disclosure. The skilled artisan will understand that the features described herein may be adapted to other types of prosthetic sockets. Hence, this disclosure and the embodiments and variations thereof are not limited to prosthetic sockets but can be utilized in any prosthetic device.

Although this disclosure describes certain exemplary embodiments and examples of a prosthetic socket, it nevertheless will be understood by those skilled in the art that the present disclosure extends beyond the specifically disclosed prosthetic socket embodiments to other alternative embodiments or uses of the disclosure and obvious modifications and equivalents thereof. It is intended that the scope of the present disclosure should not be limited by the disclosed embodiments described above, and may be extended to other prosthetic devices, and other applications that may employ the features described herein.

1.-25. (canceled)

26. A prosthetic socket adapted to adjustably fit to a residual limb, the prosthetic socket defines an axis extending centrally between proximal and distal ends of the prosthetic socket, the prosthetic socket comprising:
a socket frame including at least one strut having an elongate configuration and having a distal end connected to a distal base; and
a fabric sleeve including at least one fabric panel assembly configured to form a circumferential and a generally conical enclosure for receiving a residual limb, the at least one fabric panel assembly securing to the socket frame.

27. The prosthetic socket of claim 26, wherein the at least one fabric panel assembly defines a proximal circumference or circumferential segment adapted to adjust in size upon donning and to be urged to resiliently return to a predetermined configuration.

28. The prosthetic socket of claim 26, wherein the distal end of the at least one strut is pivotally connected to the distal base and adapted to rotate relative thereto at at least one pivot point.

29. The prosthetic socket of claim 28, wherein the at least one pivot point is lockable by a fastener extending through the distal base and engaging the at least one strut.

30. The prosthetic socket of claim 26, further comprising a funnel secured to the distal base.

31. The prosthetic socket of claim 30, wherein the at least one fabric panel assembly encircles the funnel such that the funnel extends within the enclosure formed by the at least one fabric panel assembly.

32. The prosthetic socket of claim 26, further comprising a distal lock connecting to the distal base.

33. The prosthetic socket of claim 26, wherein the at least one strut pivots about the axis, the enclosure is defined by the at least one fabric panel assembly extending about the axis.

34. The prosthetic socket of claim 26, wherein the at least one strut includes a first strut generally extending on a lateral side of the axis and a second strut generally extending on a medial side of the axis.

35. The prosthetic socket of claim 34, further comprising a medial piece connected to a proximal end of the first strut, the medial piece defining at least one wing extending laterally relative to the first strut.

36. The prosthetic socket of claim 35, wherein the at least one wing has a peripheral shape tapering away or reducing in height relative to the first strut.

37. The prosthetic socket of claim 35, wherein the at least one wing has a peripheral shape tapering away or reducing in height relative to the second strut.

38. The prosthetic socket of claim 35, wherein the medial piece is flexible or semi-flexible relative to the first strut, the first strut being substantially or entirely rigid.

39. The prosthetic socket of claim 38, wherein the medial piece defines a mounting portion slidably attached to the proximal end of the first strut by a channel defined by the mounting portion and configured and dimensioned to a cross-section of the first strut, the medial piece is adapted by the mounting portion to adjust in height relative to the first strut, the medial piece is secured along a plurality of locations relative to the proximal end of the first strut by at least one fastener locking the medial piece and the first strut.

40. The prosthetic socket of claim 26, wherein a first fabric panel of at least one fabric panel assembly is adapted to fit about the medial piece, the first fabric panel defining a first pocket configured and dimensioned to receive the medial piece and elastically expand a width to receive the medial piece and secure the first fabric panel at least about the medial piece.

41. The prosthetic socket of claim 26, further comprising a lateral piece connected to a proximal end of the second strut, the lateral piece defining at least one wing extending laterally relative to the second strut.

42. The prosthetic socket of claim 41, wherein the lateral piece is flexible or semi-flexible relative to the second strut, the second strut being substantially or entirely rigid.

43. The prosthetic socket of claim 41, wherein the lateral piece defines a mounting portion slidably attached to the proximal end of the second strut by a channel defined by the mounting portion and configured and dimensioned to a cross-section of the second strut, the lateral piece is adapted by the mounting portion to adjust in height relative to the second strut, the lateral piece is secured along a plurality of locations relative to the proximal end of the second strut by at least one fastener locking the lateral piece and the second strut.

44. The prosthetic socket of claim 43, wherein a second fabric panel of the at least one fabric panel assembly is adapted to fit about portion of the second strut, the second fabric panel defining a second pocket configured and dimensioned to receive a portion of the second strut.

45. The prosthetic socket of claim 43, wherein a second fabric panel of the at least one fabric panel assembly is adapted to fit about the lateral piece, the second fabric panel defining a third pocket configured and dimensioned to receive the lateral piece and elastically expand a width to receive the lateral piece and secure the second fabric panel at least about the lateral piece.
